(19) United States
(12) Patent Application Publication
(10) Pub. No.: US 2019/0024020 A1
(43) Pub. Date: Jan. 24, 2019

(54) CLEANING AGENT CONTAINING PROTEASE AND AMYLASE

(71) Applicant: Henkel AG & Co. KGaA, Duesseldorf (DE)

(72) Inventors: Detlef Buisker, Essen (DE); Marianne Schmeling, Korschenbroich (DE); Johannes Zipfel, Amsterdam (NL); Timothy O'Connell, Landsberg am Lech (DE)

(73) Assignee: Henkel AG & Co. KGaA, Duesseldorf (DE)

(21) Appl. No.: 16/135,683

(22) Filed: Sep. 19, 2018

Related U.S. Application Data
(63) Continuation of application No. PCT/EP2017/056656, filed on Mar. 21, 2017.

(30) Foreign Application Priority Data
Mar. 22, 2016 (DE) 10 2016 204 061.8

(51) Int. Cl. C11D 3/386; C12N 9/28; C12N 9/64

(52) U.S. Cl. CPC C11D 3/386; C12N 9/2417; C12N 1/20; C12Y 302/01001; C12Y 304/21; C12N 9/6424

(57) ABSTRACT

A cleaning agent, preferably a dishwasher detergent, preferably a hand dishwashing detergent, having at least one protease and at least one amylase as defined herein. The invention also relates to the use of the type of cleaning agent for cleaning solid surfaces and to a method for cleaning hard surfaces using the described cleaning agent. The invention further relates to the use of a specific enzyme combination as defined herein for improving the cleaning action of a cleaning agent.

### FIELD OF THE INVENTION

The present invention relates to a cleaning agent, preferably a dishwashing detergent, more preferably a hand dishwashing detergent, which contains at least one protease and at least one amylase, as defined herein, the use of a cleaning agent of this kind for cleaning solid surfaces, and a method for cleaning hard surfaces using the described cleaning agent. The invention lastly also relates to the use of a special enzyme combination, as defined herein, for improving the cleaning performance of a cleaning agent.

### BACKGROUND OF THE INVENTION

The most important criterion when cleaning hard surfaces, such as in particular when dishwashing, is the cleaning performance on various stains, which are produced in particular in the form of food residues. Even though the cleaning performance of dishwashing detergents used nowadays is generally high, there is however the problem, which is also due to the overall trend towards using increasingly low temperatures for dishwashing, that many conventional dishwashing detergents have insufficient cleaning performance on stubborn stains. Insufficient cleaning performance of this kind and the ensuing insufficient cleaning of the dishes leads to user dissatisfaction and to stains of this kind being pretreated by the user, which in turn increases water and energy consumption. There is therefore a general need for dishwashing detergents which still have good cleaning performance on stubborn stains, without thereby reducing the existing good cleaning performance on other stains.

### BRIEF SUMMARY OF THE INVENTION

The object of the present invention is therefore to provide a cleaning agent, preferably a dishwashing detergent, more preferably a hand dishwashing detergent, which has increased cleaning performance on stains of this kind, without the cleaning performance on other stains being reduced.

It has now surprisingly been determined that using a combination of a particular protease with a particular amylase synergistically improves the cleaning performance of corresponding cleaning agents, preferably a dishwashing detergent, more preferably a hand dishwashing detergent, on enzyme-sensitive stains.

In a first aspect, the present invention is therefore directed to a cleaning agent (for hard surfaces), in particular a dishwashing detergent, preferably a hand dishwashing detergent, which comprises at least one protease and at least one amylase,
a) the amylase being a hybrid amylase from the mature α-amylase from Bacilllus sp. no. 707 and the mature α-amylase from Bacillus amyloliquefaciens or a functional fragment or a variant thereof, which has an amino acid sequence which is at least 80%, preferably at least 90%, in particular 100%, identical to the amino acid sequence specified in SEQ ID NO:1 over the total length thereof; and
b) the at least one protease comprising the mature alkaline protease from Bacillus lentus DSM 5483 or a functional fragment or a variant thereof and having an amino acid sequence which is at least 80%, preferably at least 90%, in particular 100%, identical to the amino acid sequence specified in SEQ ID NO:2 over the total length thereof and which optionally has at least one amino acid substitution at one, two, three, or four of the following positions 3, 4, 99, and 199 using the numbering according to SEQ ID NO:2.

When the agent is used, a combination of this kind significantly increases cleaning performance on stubborn stains, in particular on stains containing starch.

A further subject matter of the present invention relates to the use of a cleaning agent described herein, preferably a dishwashing detergent, more preferably a machine dishwashing detergent, in a cleaning process, preferably a dishwashing process, more preferably to the use thereof for improving the cleaning performance, in particular the cleaning performance on enzyme-sensitive stains, on hard surfaces, in particular dishes when they are being cleaned, more particularly stubborn stains, inter alia also at temperatures which are lower than the temperatures usually used.

A further subject matter of the present invention relates to a cleaning method, preferably a dishwashing method, in which a cleaning agent described herein, preferably a dishwashing detergent, is used in particular for the purpose of improving the cleaning performance on enzyme-sensitive stains. In various embodiments of the invention, temperatures are used in the dishwashing method that are lower than the temperatures usually used.

A further subject matter of the invention relates to the use of the enzyme combination described herein for improving the cleaning performance of a cleaning agent, in particular a dishwashing detergent.

“Low temperatures” or “temperatures which are lower than the temperatures usually used”, as used herein in connection with dishwashing methods, preferably refers to temperatures below 60° C., in particular below 55° C., even more preferably 50° C. or lower, particularly preferably 45° C. or lower, and most preferably 40° C. or lower.

These and other aspects, features, and advantages of the invention will become apparent to a person skilled in the art through the study of the following detailed description and claims. Any feature from one aspect of the invention can be used in any other aspect of the invention. Furthermore, it will readily be understood that the examples contained herein are intended to describe and illustrate but not to limit the invention and that, in particular, the invention is not limited to these examples. Unless indicated otherwise, all percentages indicated are percent by weight. Numerical ranges that are given in the format “from x to y” include the cited values. If several preferred numerical ranges are given in this format, it is self-evident that all ranges that result from the combination of the various endpoints are also included.

“Variants”, as used herein in connection with enzymes, refers to natural or artificially created variations of an enzyme which have a modified amino acid sequence by comparison with the reference form. A variant of this kind may have individual or a plurality of point mutations, in particular one or more point mutations, i.e. substitutions of an amino acid that naturally occurs at the corresponding position with another, insertions (inserting one or more amino acids) and/or deletions (removing one or more amino acids). Variants of this kind preferably have at least 50, more preferably 60 or more, even more preferably 70, 80, 90, 100% or more of the enzyme activity of the reference form. In various embodiments, a variant of this kind has an amino acid sequence which is at least 70, preferably 75, 80, 85, 90, 95, 96, 97, 98 or 99% identical to the sequence being used as the reference over the total length thereof. The variants are preferably the same length as the reference sequence. Variants may be distinguished from the reference form by improved properties, such as higher enzyme activity, greater stability, changed substrate specificity, etc. Only those variants which have enzymatic activity are used. “Enzymatic activity”, as used in this context, means in particular that the corresponding enzymes have at least 50%, preferably at least 90%, of the catalytic activity of the reference enzyme thereof.

“Fragment”, as used herein in connection with enzymes, relates to polypeptides which, by comparison with the reference enzyme N terminal and/or C terminal, have been shortened by one or more amino acids in each case. Only those fragments which have enzymatic activity are used. “Enzymatic activity”, as used in this context, means in particular that the corresponding enzymes have at least 50%, preferably at least 90%, of the catalytic activity of the reference enzyme thereof.

The identity of nucleic acid sequences or amino acid sequences is determined by comparing the sequences. This sequence comparison is based on the BLAST algorithm, which is found in the prior art and conventionally used (cf. for example Altschul, S. F., Gish, W., Miller, W., Myers, E. W. & Lipman, D. J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs;” Nucleic Acids Res., 25, pages 3389-3402), and is principally carried out by similar sequences of nucleotides or amino acids in the nucleic acid sequences or amino acid sequences being assigned to one another. Assigning the relevant positions in a table is referred to as alignment. Another algorithm that is available in the prior art is the FASTA algorithm.

A comparison of this kind also provides information on the similarity of the compared sequences to one another. This is usually expressed in an identity percentage, i.e. the proportion of identical nucleotides or amino acid functional groups in positions that are the same or correspond to one another in an alignment. In amino acid sequences, the broader term of homology takes into consideration preserved amino acid exchanges, i.e. amino acids that have similar chemical activity, because these usually exert similar chemical activities within the protein. The similarity of the compared sequences can therefore also be expressed as a homology percentage or similarity percentage. Identity and/or homology values can be established over whole polypeptides or genes or just over individual regions. Homologous or identical regions of different nucleic acid sequences or amino acid sequences are therefore defined by agreements in the sequences. Regions of this kind often have identical functions. They can be small and only comprise a few nucleotides or amino acids. Often, small regions of this kind perform functions that are essential to the total activity of the protein. It can therefore be useful for sequence agreements to only refer to individual, optionally small, regions. Unless indicated otherwise, identity or homology values in the present application refer to the total length of the nucleic acid sequence or amino acid sequence specified in each case.

The amylases used are alkaline α-amylases. They act as hydrolases and cleave the α(1-4)-glycosidic bond from polysaccharides, in particular starches such as amylose, and thereby cause the degradation of starch-containing stains on the item to be cleaned. This produces dextrins and, as a result, maltose, glucose, and branched oligosaccharides, as cleavage products. Their optimum pH is usually notably alkaline.

Within the meaning of the present invention, an amylase which comprises an amino acid sequence that is at least 80% identical to the amino acid sequence specified in SEQ ID NO:1 over the total length thereof is used as the amylase.

Further variants which can be used are those which have an amino acid sequence that is at least 81%, 82%, 83%, 84%, 85%, 86%, 87%, 88%, 89%, 90%, 91%, 92%, 93%, 94%, 95%, 96%, 97%, 98% or 99% identical to the amino acid sequence specified in SEQ ID NO:1. Variants of this kind may include, for example, shortening the C-terminus, for example by 1 to 20 amino acids, or inserting one or more amino acids, in particular after position 182 using the numbering according to SEQ ID NO:1, for example G182GHG, or substituting one or more amino acids, in particular in the C-terminal part, i.e. the last 100 amino acids of the sequence according to SEQ ID NO:1, the enzymatic activity however being retained, i.e. the activity of the variants being at least 60% of the activity of the enzyme having the amino acid sequence of SEQ ID NO:1.

The cleaning agents described herein also contain at least one protease. The proteases are alkaline serine proteases in particular. They act as non-specific endopeptidases, i.e. they hydrolyze any acid amide bonds which are inside peptides or proteins, and thereby cause the degradation of protein-containing stains on the item to be cleaned. Their optimum pH is usually notably alkaline.

The at least one protease comprises an alkaline protease from Bacillus lentus DSM 5483 or a functional fragment or a variant thereof.

The sequence of the mature alkaline protease from Bacillus lentus DSM 5483 is specified in SEQ ID NO:2. The at least one protease comprises an alkaline protease from Bacillus lentus DSM 5483 or a functional fragment or a variant thereof, which has an amino acid sequence which is at least 80%, preferably at least 90%, in particular 100%, identical to the amino acid sequence specified in SEQ ID NO:2 over the total length thereof and which optionally has at least one amino acid substitution at one, two, three, or four of the following positions 3, 4, 99, and 199 using the numbering according to SEQ ID NO:2. Proteases which have an amino acid substitution at two, preferably three or more, in particular four, of the above-mentioned positions are preferably used.

Further variants which can be used are those which have an amino acid sequence that is at least 81%, 82%, 83%, 84%, 85%, 86%, 87%, 88%, 89%, 90%, 91%, 92%, 93%, 94%, 95%, 96%, 97%, 98%, or 99% identical to the amino acid sequence specified in SEQ ID NO:2, and which have at least one amino acid substitution at one of the following positions 3, 4, 99, and 199.

A protease of this kind particularly preferably has an amino acid sequence which is at least 80% identical to the amino acid sequence specified in SEQ ID NO:2 over the total length thereof and which has the amino acid substitutions R99E or R99D, and optionally additionally at least one or two, preferably all three, of the amino acid substitutions S3T, V4I, and V199I, using the numbering according to SEQ ID NO:2.

A protease of this kind preferably has an amino acid sequence which has at least one of, preferably a plurality of, in particular each of, the following amino acid substitutions R99E/R99D, S3T, V4I, and/or V199I using the numbering according to SEQ ID NO:2, and which is at least 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, and 99%, in particular 100%, identical to the amino acid sequence specified in SEQ ID NO:2 over the total length thereof at all other points. Particularly preferred is a protease which has an amino acid sequence that can be obtained, proceeding from the amino acid sequence having SEQ ID NO:2, by one or more of the amino acid substitutions R99E/R99D, S3T, V4I, and V199I using the numbering according to SEQ ID NO:2. A protease of this kind may have the amino acid sequence specified in SEQ ID NO:3.

In various embodiments, these combinations of amylase and protease are used in a mass ratio, based on active protein, of from 10:1 to 1:10, preferably from 5:1 to 1:5, particularly preferably from 3:1 to 1:3, even more preferably from 1:1 to 1:3, most preferably 1:2.

In various embodiments, the at least one amylase is contained in the agent according to the invention in amounts of from 0.01 to 2.0 wt. %, preferably from 0.05 to 1.5 wt. %, even more preferably from 0.1 to 1.0 wt. %, based on active protein and the total weight of the agent.

In various embodiments, the at least one protease is contained in the agent according to the invention in amounts of from 0.01 to 2.0 wt. %, preferably from 0.05 to 1.5 wt. %, most preferably from 0.1 to 1.0 wt. %, based on active protein and the total weight of the agent.

Surprisingly, the combinations described herein of particular amylases and proteases display the property of improving the performance of the cleaning agent, preferably the dishwashing detergent, in that they lead to synergistically improved cleaning performance on stubborn stains.

In this case, improvement in cleaning performance is usually understood to mean that, when the cleaning agents described herein, in particular the dishwashing detergents, are used, the removal of stains on hard surfaces, in particular dishes, while they are being cleaned is greatly improved by comparison with the use of cleaning agents, preferably dishwashing detergents, which do not contain the enzyme combinations described herein.

The enzymes to be used can also be formulated together with accompanying substances, for example from fermentation, or with stabilizers. In liquid formulations, the enzymes are preferably used as an enzyme liquid formulation or enzyme liquid formulations.

The protein concentration can be determined with the aid of known methods, for example the BCA method (bicinchoninic acid; 2,2′-biquinoline-4,4′-dicarboxylic acid) or the Biuret method. In this regard, the active protein concentration is determined by titrating the active centers while using a suitable irreversible inhibitor (for proteases, for example, phenylmethylsulfonyl fluoride (PMSF)) and determining the residual activity (cf. M. Bender et al., J. Am. Chem. Soc. 88, 24 (1966), pages 5890-5913).

Enzymes, in particular the amylases and proteases described herein, can be protected, especially during storage, against damage, for example inactivation, denaturing, or decomposition caused, for example, by physical influences, oxidation, or proteolytic cleavage. If obtained microbially, it is particularly preferred that proteolysis be inhibited. The described agents can contain stabilizers for this purpose.

Cleaning-active enzymes are usually not made available in the form of the pure protein, but rather in the form of stabilized, storable, and transportable preparations. These ready-made preparations include, for example, the solid preparations obtained through granulation, extrusion, or lyophilization or, in particular in the case of liquid or gel-like agents, solutions of the enzymes, advantageously maximally concentrated, low-moisture, and/or supplemented with stabilizers or other adjuvants.

Alternatively, the enzymes can also be encapsulated for both the solid and liquid dosage form, for example through spray-drying or extrusion of the enzyme solution together with a preferably natural polymer, or in the form of capsules, for example those in which the enzymes are enclosed in a set gel, or in those of the core-shell type in which an enzyme-containing core is coated with a water-, air-, and/or chemical-impermeable protective layer. In the case of overlaid layers, other active ingredients, such as stabilizers, emulsifiers, pigments, bleaching agents, or dyes, can be additionally applied. Capsules of this kind are applied using inherently known methods, for example through shaking or roll granulation or in fluidized bed processes.

Granulates of this kind are advantageously low in dust, for example due to the application of polymeric film-formers, and stable in storage due to the coating.

Moreover, it is possible to formulate two or more enzymes together, such that a single granulate has a plurality of enzyme activities.

### DETAILED DESCRIPTION OF THE INVENTION

As can be seen from the preceding remarks, the enzyme protein forms only a fraction of the total weight of conventional enzyme preparations. Enzyme preparations that are preferably used contain between 0.1 and 40 wt. %, preferably between 0.2 and 30 wt. %, particularly preferably between 0.4 and 20 wt. %, and in particular between 0.8 and 15 wt. % of the enzyme protein. The described agents therefore contain enzyme preparations of this kind preferably in an amount such that the active protein concentrations specified above can be achieved.

The cleaning agents described herein, in particular the preferred dishwashing detergents, can be solid or liquid, and can in particular be present as homogenous solutions or suspensions.

The agents described herein may contain surfactants, the group of surfactants including the non-ionic, anionic, cationic, and amphoteric surfactants.

All non-ionic surfactants that are known to a person skilled in the art can be used as non-ionic surfactants. Suitable non-ionic surfactants include, for example, alkyl glycosides of the general formula RO(G)x, in which R corresponds to a primary straight-chain or methyl-branched aliphatic functional group, in particular an aliphatic functional group that is methyl-branched in the 2 position, having 8 to 22, preferably 12 to 18, C atoms, and G is the symbol that represents a glycose unit having 5 or 6 C atoms, preferably glucose. The degree of oligomerization x, which indicates the distribution of monoglycosides and oligoglycosides, is any number between 1 and 10; preferably, x is from 1.2 to 1.4.

Another class of non-ionic surfactants that can preferably be used, which can be used either as a sole non-ionic surfactant or in combination with other non-ionic surfactants, are alkoxylated, preferably ethoxylated or ethoxylated and propoxylated fatty acid alkyl esters, preferably having 1 to 4 carbon atoms in the alkyl chain.

Additional suitable surfactants are the polyhydroxy fatty acid amides that are known as PHFAs.

Low-foaming non-ionic surfactants can be used as preferred surfactants. Especially preferably, the cleaning agents, preferably dishwashing detergents, contain non-ionic surfactants from the group of the alkoxylated alcohols. Non-ionic surfactants that are preferably used are alkoxylated, advantageously ethoxylated, in particular primary alcohols having preferably 8 to 18 C atoms and, on average, from 1 to 12 moles of ethylene oxide (EO) per mole of alcohol, in which the alcohol functional group can be linear or preferably methyl-branched in the 2 position, or can contain linear and methyl-branched functional groups in admixture, as are usually present in oxo alcohol functional groups. In particular however, alcohol ethoxylates having linear functional groups from alcohols of native origin having 12 to 18 carbon atoms, for example from coconut, palm, tallow fatty or oleyl alcohol, and an average of from 2 to 8 EO per mole of alcohol are preferred. Examples of preferred ethoxylated alcohols are C₁₂₋₁₄ alcohols having 3 EO or 4 EO, C₉₋₁₁ alcohols having 7 EO, C₁₃₋₁₅ alcohols having 3 EO, 5 EO, 7 EO, or 8 EO, C₁₂₋₁₈ alcohols having 3 EO, 5 EO, or 7 EO, and mixtures thereof, such as mixtures of C₁₂₋₁₄ alcohol having 3 EO and C₁₂₋₁₈ alcohol having 5 EO. The degrees of ethoxylation specified represent statistical averages that can correspond to an integer or a fractional number for a specific product. Preferred alcohol ethoxylates have a narrowed homolog distribution (narrow range ethoxylates, NRE). In addition to these non-ionic surfactants, fatty alcohols having more than 12 EO can also be used. Examples of these are tallow fatty alcohols having 14 EO, 25 EO, 30 EO, or 40 EO.

In various preferred embodiments, the agents of the invention contain at least one amine oxide as a non-ionic surfactant. In principle, all the amine oxides found in the prior art for this purpose, i.e. compounds that have the formula R¹R²R³NO, where each of R², and R³, independently of one another, is an optionally substituted, for example hydroxy-substituted, C₁-C₃₀ hydrocarbon chain, can be used in this regard. Amine oxides that are particularly preferably used are those in which R¹ is C₁₂-C₁₈ alkyl and R² and R³ are each, independently of one another, C₁-C₄ alkyl, in particular C₁₂-C₁₈ alkyl dimethyl amine oxides. Examples of representatives of suitable amine oxides are N-coconut alkyl-N,N-dimethyl amine oxide and N-tallow alkyl-N,N-dihydroxyethyl amine oxide. C₁₂-C₁₈ alkyl dimethyl amine oxides or mixtures which also contain C₁₆ and C₁₈ alkyl dimethylamine oxides are preferred over C₁₂-C₁₄ alkyl dimethylamine oxides. Similarly, amine oxides in which R¹ also comprises C₁₆ and C₁₈ functional groups are generally preferred over those in which R¹ only comprises C₁₂₋₁₄ functional groups. In various embodiments, the amine oxides used therefore contain at least 20 wt. %, preferably at least 30 wt. %, even more preferably at least 40 wt. %, most preferably at least 50 wt. %, 60 wt. %, 70 wt. %, or 80 wt. %, of C₁₆₋₁₈ amine oxides, based on the total weight of the amine oxides. In embodiments of this kind, the remainder of the amine oxides may consist of C₁₂₋₁₄ amine oxides. The at least one amine oxide is preferably used in an amount of from 0.5 to 10.0 wt. %, even more preferably from 0.8 to 8.0 wt. %, based on the total weight of the agent.

In various preferred embodiments, at least one anionic surfactant is also used. These include in particular alkylbenzene sulfonates, (fatty) alkyl sulfates, (fatty) alkyl ether sulfates, and alkane sulfonates. The cleaning agents of the invention preferably contain at least one anionic surfactant from the group of alkyl ether sulfates. Preferred alkyl ether sulfates are those of formula (I)

R¹—O—(AO)ₙ—SO₃⁻X⁺ (I)

In this formula (I), R¹ represents a linear or branched, substituted or unsubstituted alkyl functional group, preferably a linear, unsubstituted alkyl functional group, particularly preferably a fatty alcohol functional group. Preferred functional groups R¹ are selected from decyl, undecyl, dodecyl, tridecyl, tetradecyl, pentadecyl, hexadecyl, heptadecyl, octadecyl, nonadecyl, and eicosyl functional groups and mixtures thereof, the representatives which have an even number of C atoms being preferred. Particularly preferred functional groups R¹ are derived from C₁₂-C₁₈ fatty alcohols, for example from coconut fatty alcohol, tallow fatty alcohol, lauryl alcohol, myristyl alcohol, cetyl alcohol or stearyl alcohol or from C₁₀-C₂₀ oxo alcohols. X represents a monovalent cation or the n-th part of an n-valent cation, in this case the alkali metal ions, which include Na⁺ or K⁺, being preferred, Na⁺ being most preferred. Further cations X⁺ can be selected from NH₄⁺, ½ Zn²⁺, ½ Mg²⁺, ½ Ca²⁺, ½ Mn²⁺, and mixtures thereof.

AO represents an ethylene oxide (EO) group or propylene oxide (PO) group, preferably an ethylene oxide group. The index n represents an integer from 1 to 50, preferably from 1 to 20, and in particular from 2 to 10. More particularly preferably, n represents the numbers 2, 3, 4, 5, 6, 7, or 8.

Fatty alcohol ether sulfates of formula I-1

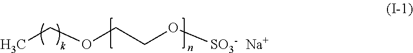

are preferred, where k=11 to 19 and n=2, 3, 4, 5, 6, 7, or 8. Particularly preferred representatives are Na—C₁₂₋₁₄ fatty alcohol ether sulfates having 2 EO (k=11 to 13, n=2 in formula II-1).

The alkyl ether sulfates are preferably used in an amount of from 3.0 to 30.0 wt. %, even more preferably from 4.0 to 25.0 wt. %, most preferably from 4.0 to 18.0 wt. %, based on the total weight of the agent.

Lastly, in various preferred embodiments the cleaning agents of the invention also contain at least one betaine. Suitable betaines are those of formula (Rⁱⁱⁱ)(R^{iv})(R^{v})N⁺CH₂COO⁻, in which Rⁱⁱⁱ denotes an alkyl functional group, which is optionally interrupted by heteroatoms or heteroatom groups, such as for example O, S, NH, C(O), C(O)NH, or C(O)O, having 8 to 25, preferably 10 to 21, carbon atoms, and R^{iv} and R^{v} denote identical or different alkyl functional groups having 1 to 3 carbon atoms. C₁₀-C₁₈ alkyl dimethyl carboxymethyl betaine and C₁₁-C₁₇ alkylamidopropyl dimethyl carboxymethyl betaine are particularly preferred. The at least one betaine is preferably used in an amount of from 0.9 to 15.0 wt. %, even more preferably from 1.0 to 12.0 wt. %, most preferably from 1.5 to 8.0 wt. %, based on the total weight of the agent.

Moreover, further surfactants can be used in the agents of the invention, in addition to the surfactants mentioned above. Suitable surfactants, for example, are surfactants from the group of alkylbenzene sulfonates, alkyl sulfonates, alkyl ester sulfonates, secondary alkane sulfonates, fatty alcohol alkoxylates, alkyl glycosides, alkoxylated fatty acid alkyl esters, fatty acid alkanolamides, hydroxy mixed ethers, sorbitan fatty acid esters, polyhydroxy fatty acid amides, and alkoxylated alcohols. Surfactants of this kind are known in the prior art.

In various embodiments, the total amount of the surfactants, based on the weight of the agent, is from 4 to 45 wt. %, preferably from 6 to 40 wt. %, even more preferably from 10 to 32 wt. %.

In one embodiment, the cleaning agents according to the present invention are liquid and contain water as a main solvent, i.e. the cleaning agents are aqueous. The water content of the aqueous agent according to the invention is usually from 15 to 95 wt. %, preferably from 20 to 90 wt. %. In various embodiments, the water content is more than 5 wt. %, preferably more than 15 wt. %, and particularly preferably more than 25 wt. %, in each case based on the total amount of cleaning agent.

In addition, non-aqueous solvents may be added to the cleaning agent. Suitable non-aqueous solvents include monovalent or polyvalent alcohols, alkanol amines or glycol ethers, if they can be mixed with water in the stated concentration range. Preferably, the solvents are selected from ethanol, n-propanol, i-propanol, butanols, glycol, propanediol, butanediol, methylpropanediol, glycerol, diglycol, propyl diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol monomethyl ether, dipropylene glycol monoethyl ether, methoxytriglycol, ethoxytriglycol, butoxytriglycol, 1-butoxyethoxy-2-propanol, 3-methyl-3-methoxybutanol, propylene-glycol-t-butylether, di-n-octylether, and mixtures of these solvents.

The one or the plurality of non-aqueous solvents is/are usually contained in an amount of from 0.1 to 10 wt. %, preferably from 1 to 8 wt. %, based on the total composition.

In addition to the previously mentioned components, the cleaning agents according to the invention may contain further ingredients which further improve the practical and/or aesthetic properties of the cleaning agent. These include, for example, additives for improving the flow and drying behavior, for adjusting the viscosity and/or for stabilization, and further auxiliaries and additives commonly found in cleaning agents, such as UV stabilizers, perfume, pearlescing agents, dyes, corrosion inhibitors, preservatives, bitterns, organic salts, disinfectants, structuring polymers, defoamers, encapsulated ingredients (e.g. encapsulated perfume), pH adjusting agents, and additives for improving or nourishing the feel of skin.

In order to further improve the flow and/or drying behavior, the agent according to the invention may contain one or more additives from the group of polymers and builders, usually in an amount of from 0.001 to 5 wt. %, preferably from 0.01 to 4 wt. %, in particular from 0.1 to 3 wt. %, particularly preferably from 0.2 to 2 wt. %, most preferably from 0.5 to 1.5 wt. %, for example 1 wt. %.

Within the meaning of the present invention, polymeric thickening agents are thickening polycarboxylates as polyelectrolytes, preferably homopolymers and copolymers of acrylic acid, in particular acrylic acid copolymers such as acrylic acid-methacrylic acid copolymers, and the polysaccharides, in particular heteropolysaccharides, and other usual thickening polymers.

Suitable polysaccharides or heteropolysaccharides are polysaccharide gums, for example gum arabic, agar, alginates, carrageenans and salts thereof, guar, gauran, tragacanth, gellan, ramsan, dextran or xanthan gum and derivatives thereof, e.g. propoxylated guar, and mixtures thereof. Alternatively or preferably in addition to a polysaccharide gum, other polysaccharide thickeners such as starches or cellulose derivatives can be used, for example starches of different origin and starch derivatives, e.g. hydroxyethyl starch, starch phosphate ester or starch acetate, or carboxymethyl cellulose or the sodium salt thereof, methyl cellulose, ethyl cellulose, hydroxyethyl cellulose, hydroxypropyl cellulose, hydroxypropyl methyl cellulose or hydroxyethyl methyl cellulose or cellulose acetate.

Acrylic acid polymers that are suitable as polymeric thickening agents are, for example, high-molecular homopolymers of acrylic acid (INCI Carbomer) that are cross-linked with a polyalkenyl polyether, in particular an allyl ether of saccharose, pentaerythritol, or propylene, which homopolymers are also referred to as carboxyvinyl polymers.

Particularly suitable polymeric thickening agents are, however, the following acrylic acid copolymers: (i) copolymers of two or more monomers from the group of acrylic acid, methacrylic acid, and the simple esters thereof (INCI Acrylates Copolymer), preferably those formed having C₁₋₄ alkanols, including for example the copolymers of methacrylic acid, butylacrylate, and methyl methacrylate (CAS 25035-69-2) or of butylacrylate and methyl methacrylate (CAS 25852-37-3); (ii) cross-linked high-molecular acrylic acid copolymers, which include for example the copolymers of C₁₀₋₃₀ alkyl acrylates that are cross-linked with an allylether of saccharose or of pentaerythritol and have one or more monomers from the group of acrylic acid, methacrylic acid, and the simple esters thereof (INCI Acrylates/C₁₀₋₃₀ Alkyl Acrylate Crosspolymer), preferably those formed having C₁₋₄ alkanols.

The content of polymeric thickening agent is usually no more than 8 wt. %, preferably between 0.1 and 7 wt. %, particularly preferably between 0.5 and 6 wt. %, in particular between 1 and 5 wt. %, and most preferably between 1.5 and 4 wt. %, for example between 2 and 2.5 wt. %, based on the total weight of the cleaning agent.

In order to stabilize the agent according to the invention, in particular when the surfactant content is high, one or more dicarboxylic acids and/or the salts thereof can be added, in particular a composition from Na salts of adipic acid, succinic acid and glutaric acid, as can be obtained for example under the trade name Sokalan® DSC. These are advantageously used in amounts of from 0.1 to 8 wt. %, preferably from 0.5 to 7 wt. %, in particular from 1.3 to 6 wt. %, and particularly preferably from 2 to 4 wt. %, based on the total weight of the cleaning agent.

However, if the use thereof can be dispensed with, the agent according to the invention is preferably free of dicarboxylic acids (or dicarboxylic acid salts).

In addition, in particular in hand dishwashing detergents and cleaning agents for hard surfaces, one or more further auxiliaries and additives, in particular UV stabilizers, perfume, pearlescing agents (INCI Opacifying Agents; for example glycol distearate, e.g. Cutina® AGS from the company Cognis, or mixtures containing this, e.g. Euperlane® from the company Cognis), dyes, corrosion inhibitors, preservatives (e.g. the technical preservative 2-bromo-2-nitropropane-1,3-diol (CAS 52-51-7), which is also referred to as Bronopol and which can be industrially obtained for example as Myacide® BT or as Boots Bronopol BT from the company Boots), organic salts, disinfectants, enzymes, pH adjusting agents, and additives for improving or nourishing the feel of skin (e.g. dermatologically effective substances such as vitamin A, vitamin B2, vitamin B12, vitamin C, vitamin E, D-Panthenol, sericerin, partial collagen hydrolysate, various partial plant protein hydrolysates, protein hydrolysate fatty acid condensate, liposomes, cholesterol, plant and animal oils such as e.g. lecithin, soybean oil, etc., plant extracts such as e.g. aloe Vera, azulene, witch hazel extracts, algae extracts, etc., allantoin, AHA complexes, glycerol, urea, quaternized hydroxyethyl cellulose) can be contained in amounts of usually no more than 5 wt. %, based on the total weight of the cleaning agent.

The pH of the agent according to the invention can be set by means of conventional pH regulators, for example acids such as mineral acids or citric acids and/or alkalis such as sodium hydroxide or potassium hydroxide, a range of from 4 to 9, preferably from 5 to 8, in particular from 5.5 to 7.5, being preferred, in particular when compatibility with the skin is desired.

In order to adjust and/or stabilize the pH, the agent according to the invention may contain one or more buffer substances (INCI Buffering Agents), usually in amounts of from 0.001 to 5 wt. %, preferably from 0.005 to 3 wt. %, in particular from 0.01 to 2 wt. %, particularly preferably from 0.05 to 1 wt. %, most preferably from 0.1 to 0.5 wt. %, for example 0.2 wt. %. Buffer substances which are simultaneously complexing agents or even chelating agents (chelators, INCI Chelating Agents) are preferred. Particularly preferred buffer substances are citric acid or citrates, in particular sodium citrates and potassium citrates, for example trisodium citrate.2 H₂O and tripotassium citrate.H₂O.

The agent according to the invention can be used for cleaning hard surfaces, in particular for cleaning dishes by hand.

The cleaning agent is prepared by means of conventional and known processes and methods.

Lastly, the present invention also relates to a method for cleaning hard surfaces, which is characterized in that an agent described herein is used in at least one method step.

A further subject matter of the invention relates to the use of an agent described herein for cleaning hard surfaces.

A further subject matter of the invention relates to the use of the enzyme combination described herein for improving the washing performance of a dishwashing detergent, preferably a hand dishwashing detergent.

All substantive matter, subject matter, and embodiments which are described for agents described herein may also be applied to the above-mentioned method and uses. At this point reference is therefore expressly made to the disclosure at the relevant point, while noting that this disclosure also applies to the above-described method and uses.

### Example 1: Formulations

Composition according to the invention (E1)

4.5% FAEOS

1.5% betaine

1.0% amine oxide

0.1% amylase (SEQ ID NO:1)

0.4% protease (SEQ ID NO:3)

up to 100% perfume, color, preservative, water

Reference composition 1 (V1)

4.5% FAEOS

1.5% betaine

1.0% amine oxide

0.21% amylase (Thermamyl 300 L)

0.43% protease (Biotouch® ROC 250 L (AB Enzymes, DE))

up to 100% perfume, color, preservative, water

Reference composition 2 (V2)

4.5% FAEOS

1.5% betaine

1.0% amine oxide

0.21% amylase (Thermamyl 300 L)

0.43% protease (Biotouch® ROC 250 L (AB Enzymes, DE))

up to 100% perfume, color, preservative, water

Reference composition 3 (V3)

4.5% FAEOS

1.5% betaine 1.0% amine oxide 0.21% amylase (Thermamyl 300 L) 0.34% protease (Purafect® Prime 4000 L (DuPont, US)) up to 100% perfume, color, preservative, water

Reference composition 4 (V4)

4.5% FAEOS

1.5% betaine

1.0% amine oxide

0.1% amylase (SEQ ID NO:1)

0.43% protease (Biotouch® ROC 250 L (AB Enzymes, DE))

up to 100% perfume, color, preservative, water

Reference composition 5 (V5)

4.5% FAEOS

1.5% betaine

1.0% amine oxide

0.21% amylase (Termamyl 300 L)

0.4% protease (SEQ ID NO:3)

up to 100% perfume, color, preservative, water

Reference composition 6 (V6)

19.1% FAEOS

7.2% betaine

4.8% amine oxide

0.3% amylase (SEQ ID NO:1)

up to 100% perfume, color, preservative, water

Reference composition 7 (V7)

19.1% FAEOS

7.2% betaine

4.8% amine oxide

0.6% amylase (SEQ ID NO:1)

up to 100% perfume, color, preservative, water

Reference composition 8 (V8)

19.1% FAEOS

7.2% betaine

4.8% amine oxide

0.3% protease (SEQ ID NO:3)

up to 100% perfume, color, preservative, water

Reference composition 9 (V9)

19.1% FAEOS

7.2% betaine

4.8% amine oxide

0.6% protease (SEQ ID NO:3)

up to 100% perfume, color, preservative, water

Composition according to the invention 2 (E2)

19.1% FAEOS

7.2% betaine

4.8% amine oxide

0.3% amylase (SEQ ID NO:1)

0.3% protease (SEQ ID NO:3) up to 100% perfume, color, preservative, water

All amounts are wt. % of active substance based on the total weight.

### Example 2: Cleaning Performance

| | Average | Average |
|---|---|---|
| | starch removed in % | protein removed in % |
| | (starch stain) | (cheese stain*) |
| E1 | 92 | 92 |
| V1 | 20 | 85 |
| V2 | 16 | 76 |
| V3 | 22 | 75 |
| V4 | 70 | 86 |
| V5 | 21 | 64 |
| V6 | | 57 |
| V7 | | 61 |
| V8 | | 37 |
| V9 | | 40 |
| E2 | | 77 |

*The cheese stain is a commercially available “4 cheese sauce”, as can be obtained from various vendors, which is deposited and burnt onto a plate or sheet metal.

It can be seen that the enzyme combination according to the invention is more effective on stains that contain starch and protein than the corresponding enzyme alone or combinations of other enzymes known in the prior art.

What is claimed is:

1. A cleaning agent, characterized in that the cleaning agent comprises at least one protease and at least one amylase,
a) the amylase being a hybrid amylase from the mature α-amylase from Bacilllus sp. no. 707 and the mature α-amylase from Bacillus amyloliquefaciens or a functional fragment or a variant thereof, which has an amino acid sequence which is at least 80% identical to the amino acid sequence specified in SEQ ID NO:1 over the total length thereof; and
b) the at least one protease comprising the mature alkaline protease from Bacillus lentus DSM 5483 or a functional fragment or a variant thereof and having an amino acid sequence which is at least 80% identical to the amino acid sequence specified in SEQ ID NO:2 over the total length thereof and which optionally has at least one amino acid substitution at one, two, three, or four of the following positions 3, 4, 99, and 199 using the numbering according to SEQ ID NO:2.

2. The cleaning agent according to claim 1, characterized in that the at least one protease has the amino acid substitution R99E or R99D, and additionally at least one or two of the amino acid substitutions S3T, V4I, and V199I.

3. The cleaning agent according to claim 2, characterized in that the at least one protease has the amino acid sequence according to SEQ ID NO:3.

4. The cleaning agent according to claim 1, characterized in that
i. the at least one amylase and the at least one protease are used in a mass ratio, based on active protein, from 10:1 to 1:10,
ii. the at least one amylase is used in amounts from 0.01 to 2.0 wt. %, based on active protein and the total weight of the agent; and/or
iii. the at least one protease is used in amounts from 0.01 to 2.0 wt. %, based on active protein and the total weight of the agent.

5. The cleaning agent according to claim 1, characterized in that the cleaning agent contains further surfactants, the total amount of the surfactants, based on the weight of the agent, being from 4 to 45 wt. %.

6. The cleaning agent according to claim 1, characterized in that the cleaning agent contains
i. at least one amine oxide, a C12-C18 alkyl dimethyl amine oxide, in an amount from 0.5 to 10.0 wt. % based on the total weight of the agent;
ii. at least one ether sulfate, in an amount from 3.0 to 30.0 wt. %, based on the total weight of the agent; and
iii. at least one betaine, in an amount from 0.9 to 15.0 wt. % based on the total weight of the agent.

7. The cleaning agent according to claim 1, characterized in that the cleaning agent contains at least one additional ingredient selected from the group consisting of additives for improving the flow and drying behavior, for adjusting the viscosity and/or for stabilization, UV stabilizers, perfume, pearlescing agents, dyes, corrosion inhibitors, preservatives, bitterns, organic salts, disinfectants, structuring polymers, defoamers, encapsulated ingredients, pH adjusting agents, and additives for improving or nourishing the feel of skin.

8. The use of a cleaning agent according to claim 1 for cleaning solid surfaces, preferably as a dishwashing detergent, particularly preferably as a hand dishwashing detergent.

9. A method for cleaning hard surfaces, characterized in that a cleaning agent, according to claim 1 is used in at least one method step.

10. The dishwashing detergent or the hand dishwashing detergent, according to claim 1, comprising at least one protease and at least one amylase,
a) the amylase being a hybrid amylase from the mature α-amylase from Bacilllus sp. no. 707 and the mature α-amylase from Bacillus amyloliquefaciens or a functional fragment or a variant thereof, which has an amino acid sequence which is at least 90% identical to the amino acid sequence specified in SEQ ID NO:1 over the total length thereof; and
b) the at least one protease comprising the mature alkaline protease from Bacillus lentus DSM 5483 or a functional fragment or a variant thereof and having an amino acid sequence which is at least 90% identical to the amino acid sequence specified in SEQ ID NO:2 over the total length thereof and which optionally has at least one amino acid substitution at one, two, three, or four of the following positions 3, 4, 99, and 199 using the numbering according to SEQ ID NO:2.

11. The dishwashing detergent or the hand dishwashing detergent, according to claim 1, comprising at least one protease and at least one amylase,
a) the amylase being a hybrid amylase from the mature α-amylase from Bacilllus sp. no. 707 and the mature α-amylase from Bacillus amyloliquefaciens or a functional fragment or a variant thereof, which has an amino acid sequence which is at least 100% identical to the amino acid sequence specified in SEQ ID NO:1 over the total length thereof; and
b) the at least one protease comprising the mature alkaline protease from Bacillus lentus DSM 5483 or a functional fragment or a variant thereof and having an amino acid sequence which is at least 100% identical to the amino acid sequence specified in SEQ ID NO:2 over the total length thereof and which optionally has at least one amino acid substitution at one, two, three, or four of the following positions 3, 4, 99, and 199 using the numbering according to SEQ ID NO:2.

12. The cleaning agent according to claim 2, characterized in that the at least one protease has the amino acid substitution R99E or R99D, and additionally all three of the amino acid substitutions S3T, V4I, and V199I.

13. The cleaning agent according to claim 4, characterized in that
i. the at least one amylase and the at least one protease are used in a mass ratio, based on active protein, from 5:1 to 1:5.
ii. the at least one amylase is used in amounts from 0.05 to 1.5 wt. %, based on active protein and the total weight of the agent; and/or
iii. the at least one protease is used in amounts from 0.05 to 1.5 wt. %, based on active protein and the total weight of the agent.

14. The cleaning agent according to claim 4, characterized in that the at least one amylase and the at least one protease are used in a mass ratio, based on active protein, from 3:1 to 1:3.

15. The cleaning agent according to claim 4, characterized in that the at least one amylase and the at least one protease are used in a mass ratio, based on active protein, from 1:1 to 1:3.

16. The cleaning agent according to claim 4, characterized in that the at least one amylase and the at least one protease are used in a mass ratio, based on active protein, 1:2.

17. The cleaning agent according to claim 5, characterized in that the cleaning agent contains further surfactants, the total amount of the surfactants, based on the weight of the agent, being from 6 to 40 wt. %.

18. The cleaning agent according to claim 5, characterized in that the cleaning agent contains further surfactants, the total amount of the surfactants, based on the weight of the agent, being from 10 to 32 wt. %.

19. The cleaning agent according to claim 6, characterized in that the cleaning agent contains
i. at least one amine oxide, a C12-C18 alkyl dimethyl amine oxide, in an amount from 0.8 to 8.0 wt. %, based on the total weight of the agent;
ii. at least one ether sulfate, in an amount from 4.0 to 25.0 wt. %, based on the total weight of the agent; and
iii. at least one betaine, in an amount from 1.0 to 12.0 wt. %, based on the total weight of the agent.


SEQUENCE LISTING (3 sequences)

SEQ ID NO: 1 - protein, 483 aa, Artificial
  Hybrid-Amylase derived from Bacillus sp. No. 707 and Bacillus amyloliquefaciens alkaline alpha-Amylase
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGPQHDYLDHPDVIGWTREGDSSHPKSGLATLITDGPGGSKRMYAGLKNAGETWYDITGNRSDTVKIGSDGWGEFHVNDGSVSIYVQK

SEQ ID NO: 2 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 3 - protein, 269 aa, Artificial
  Optimized B. lentus alkaline protease variant
AQTIPWGISRVQAPAAHNRGLTGSGVKVAVLDTGISTHPDLNIRGGASFVPGEPSTQDGNGHGTHVAGTIAALNNSIGVLGVAPSAELYAVKVLGADGEGAISSIAQGLEWAGNNGMHVANLSLGSPSPSATLEQAVNSATSRGVLVVAASGNSGASSISYPARYANAMAVGATDQNNNRASFSQYGAGLDIVAPGVNIQSTYPGSTYASLNGTSMATPHVAGAAALVKQKNPSWSNVQIRNHLKNTATSLGSTNLYGSGLVNAEAATR